(19) United States
(12) Patent Application Publication
(10) Pub. No.: US 2020/0054435 A1
(43) Pub. Date: Feb. 20, 2020

(54) GRAFT MATERIAL WITH INTERNAL FOLDS AND METHOD OF USE THEREOF

(71) Applicant: COOK MEDICAL TECHNOLOGIES LLC, BLOOMINGTON, IN (US)

(72) Inventor: Ralf Spindler, Solsberry, IN (US)

(73) Assignee: COOK MEDICAL TECHNOLOGIES LLC, BLOOMINGTON, IN (US)

(21) Appl. No.: 16/538,349

(22) Filed: Aug. 12, 2019

Related U.S. Application Data
(60) Provisional application No. 62/764,754, filed on Aug. 16, 2018.

(51) Int. Cl. A61F 2/07; A61L 31/06

(52) U.S. Cl. CPC A61F 2/07; A61L 31/06; A61F 2002/047; A61F 2210/0014; A61F 2250/0067; A61F 2210/0057

(57) ABSTRACT

The present disclosure relates to a layered graft material and to implantable medical devices including such a graft material. In one embodiment, the device is a stent-graft. In other embodiments, the invention relates to methods of manufacturing and using such devices. In one embodiment the graft is formed of at least three layers, including a folded central layer.

### PRIORITY CLAIM

This invention claims the benefit of priority of U.S. Provisional Application Ser. No. 62/764,754, entitled “Graft Material with Internal Folds and Method of Use Thereof,” filed Aug. 16, 2018, the disclosure of which is hereby incorporated by reference in its entirety.

### TECHNICAL

The present disclosure relates to a layered graft material including a layer having folded regions and to implantable medical devices including such a material. In one embodiment, the device is a stent-graft. In other embodiments, the invention relates to methods of using and manufacturing such devices. In one embodiment the graft is formed of at least three layers, including a folded central layer.

### BACKGROUND

Implantable medical devices, particularly endoluminally deployable medical devices, are known for a variety of medical applications including the treatment of aneurysms. Aneurysms occur in blood vessels at sites where, due to age, disease or genetic predisposition, the strength or resilience of the vessel wall is insufficient to prevent ballooning or stretching of the wall as blood flows therethrough. If the aneurysm is left untreated, the blood vessel wall may expand to a point at which rupture occurs, often leading to death.

To prevent rupturing of an aneurysm, such as an abdominal aortic aneurysm, a stent graft may be introduced into a blood vessel percutaneously and deployed to span the aneurysmal sac. The outer surface of each end of the stent graft is preferably sealed against the interior wall of the blood vessel at a site where the interior wall has not suffered a loss of strength or resilience. Blood flowing through the vessel is channeled through the hollow interior of the stent graft to reduce, if not eliminate, the stress on the vessel wall at the location of the aneurysmal sac. Therefore, the risk of rupture of the blood vessel wall at the aneurysmal location is significantly reduced or eliminated, and blood can pass through the vessel without interruption.

Stent grafts include a graft fabric secured to a stent. The graft is typically inserted into or pulled over the stent and attached to its structural components. Alternatively, the stent may be formed on the graft such that the individual wires of the stent are threaded through specially provided projecting fabric loops on the surface of the graft. The stent provides rigidity and structure to hold the graft open in a tubular configuration as well as the outward radial force needed to create a seal between the graft and the vessel wall. The graft provides the tubular channel for blood flow past the aneurysm and prevents blood from pressurizing the aneurysmal sac.

However, current stent graft cover material is known to sometimes exhibit a lack of stability. This may have life-threatening consequences when devices incorporating such material rupture after being implanted.

### SUMMARY

Aspects of the present invention provide a multi-layered graft material having a radially compressed state and a radially expanded state, and implantable medical devices incorporating such a graft material. In one embodiment the graft material includes first, second and third tubular layers extending longitudinally from a first end to a second end. The third layer is positioned between the first tubular layer and the second tubular layer and includes a radially unfolded region and, in the radially compressed state, a radially folded region. The third tubular layer is fixed to the first tubular layer and the second tubular layer only in the radially unfolded region.

In some embodiments, the first tubular layer and the second tubular layer are formed of a material that is radially stretchable between the compressed state and the expanded state. In other embodiments, the third tubular layer is formed of a material that is less radially stretchable between the compressed state and the expanded state than is the material forming the first and second tubular layers. In yet other embodiments, expansion of the graft from the radially compressed state to the radially expanded state causes at least a partial radial unfolding of the third tubular layer.

The first and second tubular layers may include electrospun polytetrafluoroethylene, polyurethane or fluorinated ethylene propylene. The third tubular layer may include expanded polytetrafluoroethylene, polyethylene terephthalate or c-polytetrafluroethylene.

In the radially compressed state, the third layer may include multiple radially folded regions and multiple radially unfolded regions. In such embodiments, the third tubular layer is fixed to the first tubular layer and the second tubular layer only at the radially unfolded regions. For example, in the radially compressed state, the third layer may include between 2 and 6 radially unfolded regions.

Another aspect of the present invention provides an expandable stent having a luminal and an abluminal surface and a graft material having a folded region as disclosed herein. In some embodiments, the graft material attaches to the luminal or the abluminal surface of the expandable stent.

### BRIEF DESCRIPTION OF THE DRAWINGS

Embodiments of the present invention are described below, by way of example only, with reference to the accompanying drawings, in which:

FIG. 1 is a schematic illustration showing a cross sectional view of one embodiment of a graft of the present invention. Here, the graft is shown in a radially compressed state in which the central layer of the graft includes four folded regions.

FIG. 2 is a schematic illustration showing a cross sectional view of one embodiment of a graft of the present invention. Here, the graft is shown in a partially expanded state in which the four folded regions of the central layer of the graft are partially unfolded.

FIG. 3 is a schematic illustration showing a cross sectional view of one embodiment of a graft of the present invention. Here, the graft is shown in a fully expanded state in which the four folded regions of the central layer of the graft are fully unfolded.

FIG. 4 is a graph illustrating the tensile strength of a graft material including a layer of ePTFE with and without folded regions.

### DESCRIPTION OF THE PREFERRED EMBODIMENTS

It is to be understood that the drawings are schematic only and not to scale. Often only the principal components relevant to the teachings herein are shown in the drawings, for the sake of clarity.

The term “implantable medical device” refers to a medical device that is either permanently or temporarily inserted into a patient's body for treatment of a medical condition.

The term “luminal surface,” as used herein, refers to the portion of the surface area of a medical device defining at least a portion of an interior lumen. Conversely, the term “abluminal surface,” refers to portions of the surface area of a medical device defining at least a portion of an exterior surface of the device. For example, where the medical device is a stent-graft having a stent portion with a cylindrical frame formed from a plurality of interconnected struts and bends defining a cylindrical lumen, the abluminal surface can include the exterior surface of the stent, or covering thereof, i.e. those portions of the stent or covering that are placed adjacent or in contact with the vessel wall when the stent-graft is expanded, while the luminal surface can include the interior surface of the struts and bends or covering, i.e. those portions of the device that are placed adjacent or in contact with the vessel interior when the stent-graft is expanded.

The term “therapeutic effect” as used herein means an effect which induces, ameliorates or otherwise causes an improvement in the pathological symptoms, disease progression or physiological conditions associated with or resistance to succumbing to a disorder, for example restenosis, of a human or veterinary patient. The term “therapeutically effective amount” as used with respect to a therapeutic agent means an amount of the therapeutic agent which imparts a therapeutic effect to the human or veterinary patient.

Multilayered Graft with an Internal Folded Layer

Aspects of the present invention provide multilayered graft materials and implantable medical devices incorporating such materials. In one embodiment, the graft is in the form of a multilayered graft including at least three layers. The graft may be shaped as, for example, a sheet, a tapered funnel or a tubular conduit.

In a preferred embodiment, the graft includes two outer stretchable layers, one positioned on each surface of a central layer. The central layer will generally be formed of a material that exhibits less stretchability than the outer layers and will in some preferred embodiments be formed of a material that is stronger and less subject to rupture than the outer layers. The central layer will include at least one folded region where the material of the layer is folded back onto itself. Preferably the central layer will include a series of folded regions spaced apart and arranged parallel to each other along one axis of the layer (stretchability axis). In various embodiments, the central layer includes 1, 2, 3, 4, 5, 6 or more such folded regions.

Layers formed of stretchable material are positioned on either side of the folded layer and are bonded to the folded layer to form the multi-layered graft. In one embodiment, the central layer is bonded to the stretchable layers at regions apart from the folded regions. For example, in embodiments including multiple folded regions, the layers may be bonded to each other in at least some of the regions between the folded regions. Bonding of the graft layers in such a way allows for unfolding of the folded layers when the graft is stretch in a direction perpendicular to the plane of the folded regions, i.e. when the graft is stretched along the stretchability axis.

The layers forming the graft may be attached to each other by, for example, pressing the layers together at an elevated temperature. In such a procedure, the temperature should be such that at least one of the layers undergoes at least a limited melting, resulting in a bonding of the layers. In other embodiments, the layers are attached by, for example an adhesive or by sutures or staples.

In some embodiments, the graft may include additional layers. For example, the graft may include multiple layers including folded regions. In some such embodiments, the layers containing folded regions are separated by stretchable layers. The graft may include, for example, 1, 2, 3, 4, 5 or more layers including folded regions.

Turning now to FIGS. 1 to 3, there is here illustrated a schematic illustration showing a cross sectional view of one embodiment of a graft of the present invention. In FIG. 1, graft 100 is shown in a radially compressed state in which central layer 130 includes four folded regions 140. Central layer 130 is positioned between stretchable layers 110 and 120, stretchable layer 110 on the luminal side of the central layer and stretchable layer 120 on the abluminal side of the central layer. The three layers are bonded together in at least some of the regions between folded regions 140.

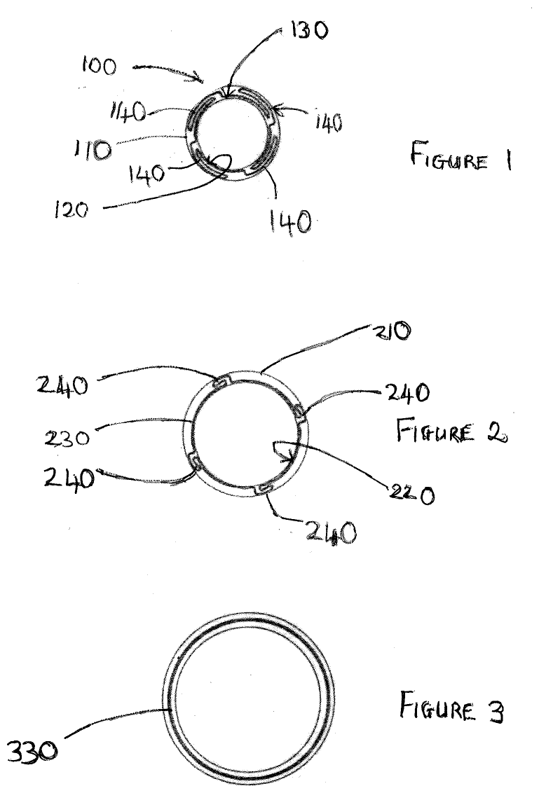

FIG. 2 is a schematic illustration showing the graft in a partially expanded state. For example, if the graft is attached to a balloon or self-expandable stent, the graft may be in this configuration when the stent is partially expanded. Here, the four folded regions 240 of central layer 230 of the graft are partially unfolded. Stretchable layers 210 and 220 accommodate the increased cross section of the graft due to their stretchability. However, in the absence of folded regions 140, central layer 230 would not have sufficient ability to stretch, possibly resulting to incomplete expansion of the stent or rupture of central layer.

FIG. 3 is a schematic illustration showing the graft in a fully expanded state in which the four folded regions of central layer 330 of the graft are fully unfolded. This state represents the maximum expansion of the graft without relying on any natural stretchability of central layer 330.

In certain embodiments, the folded layer includes, or is formed from, expanded polytetrafluoroethylene (“ePTFE”), polyethylene terephthalate or compressed polytetrafluoroethylene (“cPTFE”). In other embodiments, the stretchable layers of the graft include, or are formed from, electrospun polytetrafluoroethylene “(esPTFE”), polyurethane or fluorinated ethylene propylene. esPTFE is formed by the use of an electric force to draw charged threads of PTFE polymer solutions or polymer melts up to fiber diameters in the order of some hundred nanometers. ePTFE has a micro-structure characterized by nodes interconnected by fibrils of the polymer. The material is formed by expanding paste-formed products of a tetrafluoroethylene polymer to form a material having high porosity and high strength. Both esPTFE and ePTFE materials are commercially available in sheet form from, for example, Zeus Industrial Products, Inc., Orangeburg, S.C. 29115.

In other embodiments, the folded layer is a mesh or a braided, woven or knitted layer. The folded layer may be formed from, for example, polyether ether ketone (PEEK), Polyethylene terephthalate (PETE), ultra-high-molecular-weight polyethylene (UHMWPE), nylon, or a metallic material, such as a super-elastic nickel-titanium alloy (e.g. NITINOL), stainless steel, gold, platinum, palladium, titanium, tantalum, tungsten, molybdenum, cobalt-chromium alloy, such as L-605, MP35N, Elgiloy; nickel-chromium alloys, such as alloy 625; and niobium alloys, such as Nb-1% Zr.

### Implantable Devices Incorporating a Multilayered Graft with an Internal Folded Layer

The multilayered graft with an internal folded layer may form part of implantable medical devices such as, but not limited to, endovascular grafts, vascular grafts, stent grafts, balloon catheters, meshes, filters (e.g., vena cava filters), tissue scaffolds, myocardial plugs, valves (e.g., venous valves), pelvic implants, various types of dressings, or other known implantable devices, including flat sheet structures such as hernia patches, skin graft patches, bone stabilization devices or bandages.

The medical device may be a bifurcated integrated stent-graft, an integrated stent-graft configured for any blood vessel including coronary arteries and peripheral arteries (e.g., renal, superficial femoral, carotid, and the like), a urethral integrated stent-graft, a biliary integrated stent-graft, a tracheal integrated stent-graft, a gastrointestinal integrated stent-graft, or an esophageal integrated stent-graft, for example.

Typically, in stent-graft devices, the graft is attached to the stent portion of the devices by, for example, sutures or an adhesive, so that when the stent is expanded alter delivery to the treatment site, the fabric material contacts the vessel wall and provides support for any weakness present.

Preferably, the graft portion of the device is attached to the stent with the stent in a compressed state. In such a state, the stent is able to accept the graft in a state where the folded regions of the folded layer are fully folded. Of course, in some embodiments, the stent portion may be compressed sufficiently to accept to folded graft, the graft attached, and then the stent compressed additionally to a size small enough to fit the delivery device.

In preferred embodiments, a graft as described herein is attached to a balloon expandable or self-expanding stent to form a stent-graft device. The stent portion of the device is generally formed of at least one tubular portion and may be configured as a unitary structure or as a plurality of attached portions, for example, attached tubular portions or a plurality of interconnected struts, which may collectively define the stent portion. The tubular portion may be made from a woven or knitted structure, a laser-cut cannula, individual interconnected rings, or another pattern or design.

The stent portion may be formed from a metallic material such as stainless steel, super-elastic nickel-titanium (NITINOL), silver, platinum, palladium, gold, titanium, tantalum, iridium, tungsten, cobalt, chromium, cobalt-chromium alloy, cobalt-based alloy, nickel-based alloy or molybdenum alloy. Biodegradable metals may also be used, including, for example, a biodegradable magnesium alloy.

In other embodiments, the stent portion may my formed from a biodegradable or non-biodegradable polymeric material. Nonbiodegradable polymers that can be used include for example cellulose acetate, cellulose nitrate, silicone, polyethylene terephthalate, polyurethane, polyamide, polyester (e.g. Nylon), polyorthoester, polyanhydride, polyether sulfone, polycarbonate, polypropylene, high molecular weight polyethylene, and polytetrafluoroethylene, or mixtures of these materials. Biodegradable polymers that can be used include for instance polylactic acid (PLA), polyglycolic acid (PGA), poly(lactic-co-glycolic acid) (PLGA), polyanhydride, polycaprolactone, polyhydroxybutyrate valerate, or mixtures of these materials.

### Devices Incorporating Bioactive Agents

The grafts and implantable medical devices disclosed herein may also include a therapeutically effective amount of a bioactive agent. For example, the bioactive agent may be incorporated into the graft and/or into another component of the device. For example, in the case of stent-graft devices, the bioactive agent may be incorporated into the one or more layers of the graft. The bioactive material may be incorporated during the manufacturing process used for form the individual layers of the graft. In other embodiments, the bioactive agent may be impregnated into the graft after it has be formed by combining the individual layers.

The bioactive agent may be selected to perform a desired function upon implantation. Bioactive agents within the scope of the present embodiments include antiproliferative agents immunosuppressive agents, restenosis-inhibiting agents, anti-cancer agents, analgesics/antipyretics, anesthetics, antiasthmatics, antibiotics, antidepressants, antidiabetics, antifungal agents, antihypertensive agents, anti-inflammatories, antineoplastics, antianxiety agents, sedatives/hypnotics, antianginal agents, nitrates, antipsychotic agents, antimanic agents, antiarrhythmics, antiarthritic agents, antigout agents, thrombolytic agents, hemorheologic agents, anticonvulsants, antihistamines, agents useful for calcium regulation, antibacterial agents, antiviral agents, antimicrobials, anti-infectives, bronchodilators, steroids and hormones.

Non-limiting examples of such drugs include doxorubicin, camptothecin, etoposide, mitoxantrone, cyclosporine, epothilones, napthoquinones, 5 fluorouracil, methotrexate, colchicines, vincristine, vinblastine, gemcitabine, statins (for example atorvastatin, fluvastatin, lovastatin, pitavastatin, pravastatin, rosuvastatin and simvastatin), steroids (for example cortisteroids, prednisilone and dexamethazone) mitomycin and derivatives or analogues of these agents.

Preferred bioactive agents include restenosis-inhibiting agents, including but not limited to microtubule stabilizing agent such as paclitaxel, a paclitaxel analog, or a paclitaxel derivative or other taxane compound; a macrolide immunosuppressive agent such as sirolimus (rapamycin), pimecrolimus, tacrolimus, everolimus, zotarolimus, novolimus, myolimus, temsirolimus, deforolimus, or biolimus; an antiproliferative agent; a smooth muscle cell inhibitor; an inhibitor of the mammalian target of rapamycin (mTOR inhibitor).

Certain bioactive agents may be present in more than one polymorphic form. For example, paclitaxel may be present as at one of solid forms of amorphous paclitaxel (“aPTX”), dihydrate crystalline paclitaxel (“dPTX”) and anhydrous crystalline paclitaxel.

### Example 1—Tensile Test Determination of Multilayer Grafts Including Folded ePTFE Layers

Multi-layer composite grafts are formed by stacking alternating layers of PTFE and polyurethane and heating the stacked composite to partially melt the polyurethane to bond the layers together. Three PTFE layers are stacked each side of a central polyurethane layer with a further polyurethane layer positioned between each PTFE layer. The inner most and outer most PTFE layer on each side of the central polyurethane layer is formed of es-PTFE and the middle layer is formed of e-PTFE.

Four composite grafts are formed, the first without folds in the ePTFE layers. The second graft includes one fold of approximately 1 mm wide in each of the ePTFE layers. The ePTFE is orientated to maximize tensile strength and the folds are orientated parallel to each other perpendicular to the required direction of expansion. The third graft is similar to the second graft except that each ePTFE layer includes two folds, each approximately 1 mm wide. The fourth graft does not include an ePTFE layer.

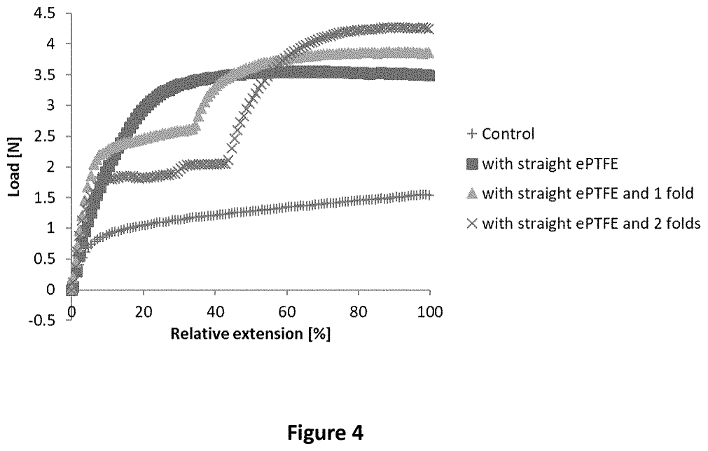

FIG. 4 shows tensile test results for each of the composite grafts. The graph shows that adding a layer of ePTFE increases the tensile strength compared to the Control. Furthermore, if the ePTFE layer has one or more folds, then, the tensile strength is further increased above a customized threshold. The curve with 1 fold shows the extension threshold at −35% and the curve with 2 folds shows the extension threshold at −44%. Below the extension threshold the material is more flexible whereas above the graft material has a higher mechanical strength, thus lowering the risk of creep rupture.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

What is claimed is:

1. A tubular graft material having a radially compressed state and a radially expanded state, the graft material comprising:
a first tubular layer extending longitudinally from a first end to a second end;
a second tubular layer extending longitudinally from the first end to the second end; and
a third tubular layer extending longitudinally from the first end to the second end,
wherein the third layer is positioned between the first tubular layer and the second tubular layer, wherein the third layer comprises a radially unfolded region and, in the radially compressed state, a radially folded region, and
wherein the third tubular layer is fixed to the first tubular layer and the second tubular layer only in the radially unfolded region.

2. The graft material of claim 1, wherein the first tubular layer and the second tubular layer are formed of a material that is radially stretchable between the compressed state and the expanded state.

3. The graft material of claim 2, wherein the third tubular layer is formed of a material that is less radially stretchable between the compressed state and the expanded state than the material forming the first and second tubular layers.

4. The graft material of claim 3, wherein expansion from the radially compressed state to the radially expanded state causes at least a partial radial unfolding of the third tubular layer.

5. The graft material of claim 1, wherein the first tubular layer and the second tubular layer comprise a material selected from the group consisting of electrospun polytetrafluoroethylene, polyurethane and fluorinated ethylene propylene.

6. The graft material of claim 5, wherein the third tubular layer comprises a material selected from the group consisting of expanded polytetrafluoroethylene, polyethylene terephthalate and c-polytetrafluroethylene.

7. The graft material of claim 1, wherein, in the radially compressed state, the third layer comprises a plurality of radially folded regions and a plurality of radially unfolded region in the radially compressed state, and wherein the third tubular layer is fixed longitudinally to the first tubular layer and the second tubular layer only at the radially unfolded regions.

8. The graft material of claim 1, wherein, in the radially compressed state, the third layer comprises between 2 and 6 radially unfolded regions.

9. The graft material of claim 8, wherein, in the radially compressed state, the third layer comprises 4 radially unfolded regions.

10. A stent graft comprising:
an expandable stent having a luminal and an abluminal surface: and
a graft material having a radially compressed state and a radially expanded state, the graft material comprising:
a first tubular layer extending longitudinally from a first end to a second end;
a second tubular layer extending longitudinally from the first end to the second end; and
a third tubular layer extending longitudinally from the first end to the second end, wherein the third layer is positioned between the first tubular layer and the second tubular layer, wherein the third layer comprises a radially unfolded region and, in the radially compressed state, a radially folded region, and wherein the third tubular layer is fixed longitudinally to the first tubular layer and the second tubular layer only in the radially unfolded region,
wherein the first layer of the graft material attaches to the luminal or the abluminal surface of the expandable stent.

11. The stent graft of claim 10, wherein the first layer of the graft material attaches to the abluminal surface of the stent.

12. The stent graft of claim 10, wherein the first tubular layer and the second tubular layer are formed of a material that is radially stretchable between the compressed state and the expanded state.

13. The stent graft of claim 10, wherein the third tubular layer is formed of a material that is less radially stretchable between the compressed state and the expanded state than the material forming the first and second tubular layers.

14. The stent graft of claim 13, wherein expansion from the radially compressed state and the radially expanded state causes at least a partial radial unfolding of the third tubular layer.

15. The stent graft of claim 10, wherein the first tubular layer and the second tubular layer comprise electrospun polytetrafluoroethylene.

16. The stent graft of claim 15, wherein the third tubular layer comprises expanded polytetrafluoroethylene.

17. The stent graft of claim 10, wherein, in the radially compressed state, the third layer comprises a plurality of radially folded regions and a plurality of radially unfolded region in the radially compressed state, and wherein the third tubular layer is fixed longitudinally to the first tubular layer and the second tubular layer only at the radially unfolded regions.

18. The stent graft of claim 8, wherein, in the radially compressed state, the third layer comprises 4 radially unfolded regions.

19. A stent graft comprising:
an expandable stent having a luminal and an abluminal surface; and
a graft material having a radially compressed state and a radially expanded state, the graft material comprising:
a first tubular layer extending longitudinally from a first end to a second end;
a second tubular layer extending longitudinally from the first end to the second end; and
a third tubular layer extending longitudinally from the first end to the second end, wherein the third layer is positioned between the first tubular layer and the second tubular layer, wherein the third layer comprises a radially unfolded region and, in the radially compressed state, a radially folded region, and wherein the third tubular layer is fixed longitudinally to the first tubular layer and the second tubular layer only in the radially unfolded region,
wherein the graft material attaches to the luminal or the abluminal surface of the expandable stent; wherein the first and the second tubular layers comprise electrospun polytetrafluoroethylene and wherein the third tubular layer comprises expanded polytetrafluoroethylene.
